(19) United States
(12) Patent Application Publication
(10) Pub. No.: US 2022/0130526 A1
(43) Pub. Date: Apr. 28, 2022

(54) ANNA (All Now Network Access)

(71) Applicant: Robert Shaun Slattery, Roland, AR (US)

(72) Inventor: Robert Shaun Slattery, Roland, AR (US)

(21) Appl. No.: 17/081,886

(22) Filed: Oct. 27, 2020

(51) Int. Cl. G16H 40/20; G16H 10/60; G06Q 30/02; G06N 3/04

(52) U.S. Cl. CPC G16H 40/20; G16H 10/60; G06Q 30/0201; G06N 3/04; G06Q 30/0283

(57) ABSTRACT

ANNA™ is an artificial intelligence solution that cognitively processes healthcare consumer demand based on an underlying link of suppliers; ANNA™ platform is used to identify demand of consumers (individuals, employers, government and commercial payers) and match with future capacity of healthcare suppliers. ANNA™ platform is based on proprietary transactional capacity standard for healthcare. ANNA™ application communicates capacity availability, scheduling and purchasing of healthcare services irrespective of the providers electronic healthcare operating system(s). The capacity standard allows for rapid packaging and exchange of data and information into ANNA™ neural networks and learning infrastructure. The networks algorithms, machine learning and feedback mechanisms efficiently connect consumers with supplier care access information. ANNA™ analytics provides additional data on costs and quality of care for consumers and suppliers at point of purchase. Access to ANNA™ analytics has secondary uses for consumers, suppliers and payors.

### CROSS REFERENCES TO RELATED APPLICATIONS

Not applicable

### STATEMENT OF FEDERALLY SPONSORED RESEARCH

The ANNA™ invention is not a product, a method, or utility of federally sponsored research or development.

### STATEMENT OF JOINT RESEARCH AGREEMENT

No parties are currently involved in a joint research agreement other than Primary Inventor 1, Robert S. Slattery and Secondary Inventor 2, Katherine L. Ball.

### NO REFERENCES

No references to “Sequence Listing” or a computer program are listed nor applicable.

### BACKGROUND

ANNA™ was invented to reduce the existing mismatch of consumer demand for healthcare services versus the capacity available from suppliers. Building facility for access and training providers is timely and expensive for our communities, societies, and taxpayers. Currently, capacity for care delivery services exists, with consumers unable to easily achieve timely access, based on their personal preferences for care. ANNA™ an artificial intelligence solution that cognitively processes healthcare consumer demand based on an underlying link of suppliers.

### BRIEF SUMMARY

ANNA™ (All Now Network Access): ANNA™ is a multifaceted cloud-based database and schema that utilizes artificial intelligence processing protocols to reimagine healthcare supplier workflows to improve consumer access to available healthcare services, including, amongst other things, availability of diagnostics, procedures, specialty care, and care management resources through interoperability computer-based methods. Fundamental to ANNA™ is a proprietary capacity transactional standard that links consumer demand to supplier's capacity. Through computer methods, access, quality and cost data, amongst other things, are presented to consumers, to best determine preferences for access options of care. The artificial intelligence platform through machine learning, sensing and adapting with disparate electronic operating systems within the healthcare domain, ANNA™ creates virtual one stop open access marketplace benefiting individuals, suppliers and payers. ANNA™ functions on the principles of an open and transparent market that strives to lower costs while improving health outcomes by maximizing access points to care resulting in key stakeholder satisfaction.

### BRIEF DESCRIPTION OF DRAWINGS

a. FIG. 1.

ANNA™ is a computerized method that utilizes a proprietary standard transactional language and format for the packaging and exchange of electronic scheduling data elements, thereby allowing the ANNA™ software application the ability to communicate capacity availability, scheduling and purchasing of healthcare services irrespective of the operating system and/or systems. Certain ANNA™ neural network capabilities such as Domain Nodes and data Synapses are be shared for multiple marketplaces, allowing for reuse of data amongst customers. ANNA™'s analytics properly and securely shared shall enable new business decisions and designs for suppliers and consumers of healthcare services. For example, a hospital (supplier) that contracts to a network (or networks) of insurers (payers) that manage care for its members (consumers) would want to share its ANNA™ data node across multiple domains nodes in order to fill excess capacity by setting upper and lower constraints based on custom pricing, bidding, reverse auction, quality outcomes and satisfaction scores schema.

The real-world implementation of ANNA™ shall result in a new industry standard for managing and facilitating scheduling transactions for suppliers and consumers within the healthcare industry. Similar to other regulatory standards for the electronic submission of Health Insurance Portability and Accountability Act (HIPM) compliant health claims and encounters, membership files, and financial transactions; ANNA™ integrates global healthcare delivery capacity by creating a compliant scheduling and capacity management standard.

Network Domain Nodes:

A supplier, client, line of business, employer group, insurer, or other entity or dataset that should be separated from other suppliers, clients, lines of business, insurer, employer groups, or other entities. Domains are configured to define where each network synapses for the domain resides (server, database type, and database name) and have certain settings (default field values, varying rules, and data paths) which are uniquely defined for each domain.

Data Synapses:

A set of related tables which can be grouped together in a single database or schema. The network synapses are physically differentiated from one another so there can be multilayered views created across the network that allows for time, cost, quality, preference, availability and price to be intelligently and automatically presented by the supplier to the consumer to effectuate real-time review and transactions based on pre-defined criteria. Certain synapses may be shared for multiple domains, allowing for reuse of data when it can be properly shared.

b. FIG. 2.

In FIG. 2, ANNA™ User Story, provides a general schema of the methods and processes of access and timely care access.

Through the world wide web, a consumer accesses ANNA™ network, meeting HIPAA, Public Law 104-191, privacy, security, technical, physical and administrative requirements. The consumer access goes through an authentication process. The consumer sets their care access preferences including but not limited to location, time, provider and network coverage for care-event, diagnostic, procedural, or specialty care. The consumer decides based on real-time capacity, the facilities and providers to best suit their timely care needs. ANNA™ AI links consumer to the suppliers through a proprietary capacity standard regardless of the providers electronic health operating system(s). The capacity standard allows for rapid packaging and exchange of data and information into ANNA™ neural networks and learning infrastructure. ANNA™ machine learning and feedback mechanisms efficiently connect consumers with supplier care access information. The networks algorithms leverage data access that put patients first, supporting the MyHealthEData Initiative and codified in CMS Rule 9115-F, the Interoperability and Patient Access final rule. In addition to Patient Access, Provider Directory, Payor-to Payor Data APIs, ANNA™ analytics will explore payor claims and clinical data, as well as provider electric health records data, including case management data from both payor and provider. ANNA™ analytics provides additional data on costs and quality of care for consumers and suppliers at point of purchase. The data will be scrutinized from governmental, non-profit and for-profit quality measurement organizations, such as the National Quality Forum, Institute for Healthcare Improvement, CMMS, The Leapfrog Group or Premier Inc. Quality data evidence-based medicine (EBM) therapies and outcomes are extracted and analyzed through ANNA™ AI.

FIG. 2 may be considered as an example of process for thousands of healthcare access scenarios. One such example may include the use of ANNA™ for a patient or their provider that has a newly identified breast lump including timely access to scheduling 3D Mammogram, arranging a breast biopsy, accessing an oncologist and/or care-management bundles. Another example may be a person determined to have a rotator cuff injury and arranging timely access for scheduling an MRI, access to a sports medicine orthopedic specialist and follow up physical therapy access.

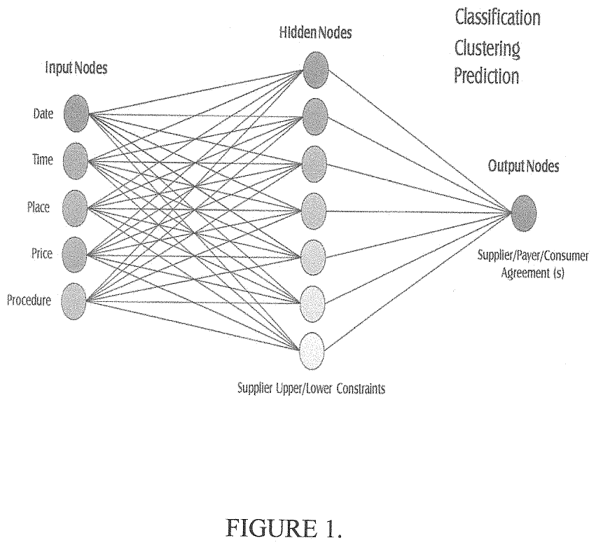

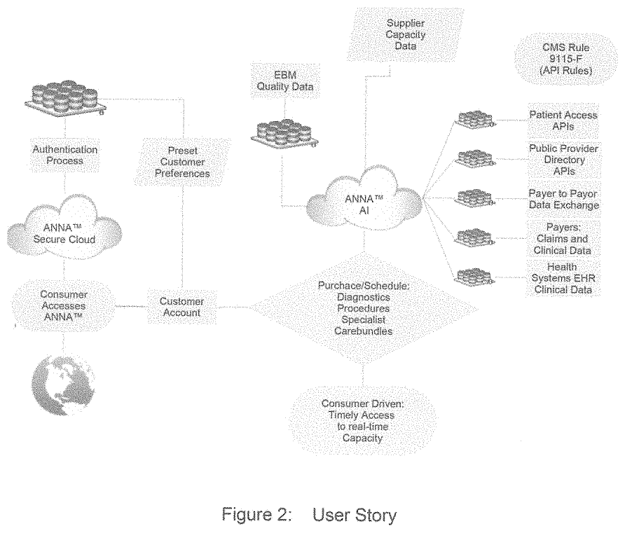

### DETAILED DESCRIPTION

Purpose: The ANNA™ computer implemented method infrastructure enhances access to healthcare in a consumer-oriented marketplace by identifying current and future capacities for diagnostics, preventive and specialty care, and procedures, amongst other things.

Structure/Operation: ANNA™ platform is based on proprietary transactional capacity standard for healthcare. ANNA™ application communicates capacity availability, scheduling and purchasing of healthcare services irrespective of the providers electronic healthcare operating system(s). The capacity standard allows for rapid packaging and exchange of data and information into ANNA™ neural networks and learning infrastructure. ANNA™'s foundation is an AI platform with advanced algorithms imbedded into distributed neural networks. ANNA™'s high impact operations shall manage a broad range of use-cases to match regional access points for healthcare delivery services with consumers needs based on current and future capacity. Its complementary cloud and edge-cloud provides mesh networking and distributed capabilities for connecting disparate disconnected healthcare scheduling systems through ANNA™s capacity standard. The networks algorithms, machine learning and feedback mechanisms efficiently connect consumers with supplier care access information. ANNA™ analytics provides additional data on costs and quality of care for consumers and suppliers at point of purchase. Access to ANNA™ analytics has secondary uses for consumers, suppliers and payors.

ANNA™ utilizes a proprietary standard transactional language and format for the packaging and exchange of electronic scheduling data elements, thereby allowing the ANNA™ software application the ability to communicate capacity availability, scheduling and purchasing of healthcare services irrespective of the operating system and/or systems. Certain ANNA™ neural network capabilities such as Domain Nodes and data Synapses may be shared for multiple marketplaces, allowing for reuse of data amongst customers. ANNA™'s analytics properly and securely shared shall enable new business decisions and designs for suppliers and consumers of healthcare services. For example, a hospital (supplier) that contracts to a network (or networks) of insurers (payers) that manage care for its members (consumers) would want to share its ANNA™ data node across multiple domains nodes in order to fill excess capacity by setting upper and lower constraints based on custom pricing, bidding, reverse auction, quality outcomes and satisfaction scores schema by creating a compliant scheduling and capacity management standard.

### SEQUENCE LISTING

Not Applicable

1. A computer implemented method, for linking healthcare consumer's access demand to available provider healthcare capacity, the method comprising: identifying and registering consumer, delivering consumer preference for care type access, utilizing analytics from an artificial intelligence-based processing model, wherein the plurality of data sources include capacity data from suppliers, cost data, quality data and application programming interface data from payors and providers and assigning options of access to consumer.

2. A method of claim 1, wherein though a virtual encounter, identifying and receiving customer identification and registration component comprising: executing a multifactor authentication process on virtual patient encounter platform, based in cloud software services for automated processing of customer information including networking services, storage services, virtualization services, operating system services and run-time services.

3. The method of claim 1, wherein the registration component includes a plurality of fields comprising: a queue name, name, schedule type, status, age and a web application and/or kiosk check-in, and/or transmittal of scanned image of federal or state ID, primary, secondary and prescription insurance data.

4. A method of claim 1, for resulting consumer preference evaluation data comprising: extracting, searching and analyzing, consumer data from an artificial intelligence-based processing model, wherein the plurality of data sources includes unique consumer driven data units and ranked settings for such units, including but not limited to facility, provider, network, location and time preferences.

5. A computer implemented method for receiving and transmitting supplier's access capacity data from a cloud-based server processing structure comprising: utilizing multiple processors, communicating with supplier's transceiver, utilizing an interoperable transactional capacity standard, linking supplier's capacity data to an artificial intelligence-based processing model.

6. A method of claim 5, wherein a transactional capacity standard comprising: extracting capacity from provider electronic health operating systems, from a proprietary standard developed for specifically for electronically exchanging capacity data, from a plurality of data sources including inpatient and outpatient facilities' capacity data, for diagnostics, procedural and provider visits types including specialty care.

7. A computer implemented method of claim 5 displaying curated capacity options for consumers, the system comprising: a database storing consumer identity data, preference data, profile data as non-transferable computer readable storage media, a controller communicably coupled to the database, a user profile component configured for assisting a consumer and configured for developing curated capacity options comprising at least one option based on the user's access preference profile.

8. A method for automated processing of healthcare application programming data (API) information, comprising: creating on a plurality of cloud server network devices on a cloud communications network, a plurality cloud hardware resources comprising in processing of API data from, Patient Access, Provider Directory, Payor-to Payor Data APIs, analytics outputs for payor claims and clinical data, as well as provider electric health records data, including case management and prior authorization criteria and data.

9. A method for claim 1, a computer implemented method for displaying healthcare costs, comprising: providing a processor configured for implementing a healthcare cost estimator, said estimator creating a cost breakdown for use in connection with a healthcare event; said processor configured for presenting a plurality capacity access options to user valued for cost.

10. The method of claim 9, wherein said component cost breakdowns comprising: evaluating pricing based on the scheduling of services according to the availability within the suppliers scheduling software.

11. The method of claim 9, wherein said availability is adjustable comprising: removing and/or adding criteria as applicable to increase utilization of supplier's capacity resources.

12. The method of claim 11, further comprising: using artificial intelligence neural network nodes that allow the supplier to develop and select criteria from a plurality of data including but not to time, place, cost and price in order to optimize utilization of resources thereby increasing capacity.

13. The method of claim 11, further comprising; using said neural network nodes to establish an algorithmic process and machine learning for the supplier to enhance decision making from data and outputs from an artificial intelligence-based processing model.

14. The method of claim 13, further comprising: providing availability of a data from numerous nodes that filter supplier decisions in a manner that a user can choose options to set a price with a plurality of option criteria including but not limited to a buy now fixed bid, reverse bid, reserve price with incremental bid minimums, that adhere to a supplier configured logic within a pricing domain and node setting.

15. The method of claim 13, further comprising: using said nodes and subsequent establishing a broader domain environment to facilitate integration and communication with the supplier community and providing access to the consumer.

16. The method of claim 1, further comprising: integrating quality data and service charts and related health management best-practice tools, including automated tools, to help a customer and supplier in managing and tracking a procedure or service.

17. The method of claim 1, further comprising: integrating supplier domains in combination of proprietary capacity that can intuitively increase utilization by engaging consumers via an information hub that facilitates improving consumer online monitoring capabilities of critical, non-static cost, pricing, quality, access availability criteria in real-time.

18. The method of claim 8, wherein said pricing model comprising: estimating and extrapolating dependencies data from healthcare suppliers and payors, including and but not limited to evidence-based authorization criteria, reference-based pricing, health insurance benefits, as well as patient and consumer experience information from supplier and industry related sources.

19. A method for claim 8, wherein said computer implemented method for displaying healthcare quality indicators, comprising providing a processor configured for analytics from an artificial intelligence-based processing model on healthcare quality data from governmental, non-profit and for-profit quality measurement organizations.

20. The method of claim 8, further compromising: monitoring the cost of an interaction, as well as cost and pricing elements therein; and providing a notification to the user (i.e., supplier, payor, or consumer) when an element in the decision node or domain occurs relative to a predetermined threshold, or certain level or range.
